(19) United States
(12) Patent Application Publication
(10) Pub. No.: US 2009/0312235 A1
(43) Pub. Date: Dec. 17, 2009

(54) Method for producing insulin in the form of an oral preparation

(75) Inventors: Andrey Vladimirovich Artamonov, Novosibirsk (RU); Petr Ivanovich Rodionov, Moskovskaya obl. (RU)

Correspondence Address: Davidson, Davidson & Kappel, LLC, 485 7th Avenue, 14th Floor, New York, NY, 10018, US

(73) Assignee: Concern O3'Company Ltd., MOSCOW (RU)

(21) Appl. No.: 12/310,892

(22) PCT Filed: Sep. 11, 2007
(86) PCT No.: PCT/RU2007/000483

§ 371 Date: Mar. 11, 2009

(30) Foreign Application Priority Data
Sep. 13, 2006 (RU) 2006132763

(51) Int. Cl. A61K 38/28; A61P 3/10

(52) U.S. Cl. 514/3

(57) ABSTRACT

The invention belongs to pharmacology and medicine, particularly, to endocrinology.

Essence of the method is addition of insulin in 1-50% aqueous solution of aqueous soluble polymer with molar mass of 0.4-40 kDa previously irradiated with ionizing radiation of 1.0-5.0 Mrad, up to final concentration of 1-10 mg/ml in polyethyleneoxide: insulin ration equal to (1-500):1 and stirring of the mixture up to obtaining of homogeneous transparent or slightly opalescent solution. This method allows to obtain the pharmaceutical composition comprising a biologically active insulin which exhibits a high hypoglycemic activity when enterally administered.

The method allows to simplify the process of obtaining of enteral insulin preparation and to increase its therapeutic efficiency.

### THE FIELD OF INVENTION

The invention belongs to pharmacology and medicine, particularly, to endocrinology and can be used for producing insulin in the form of an oral preparation.

### PRELIST TECHNIC LEVEL

Insulin is the polypeptide hormone with molar mass about 6000. It influences all the types of organism metabolism: increases penetration of glucose in organism tissues and its use by them, decreases glycogen content in liver and increases its quantity in muscles, increases protein synthesis intensity etc.

The main route for insulin administration in organism is subcutaneous or intramuscular injections of preparation. Attempts of insulin preparation by oral (by mouth) route, being the most physiological and convenient for patients, met with failure, since insulin is hydrolyzed easily by digestive enzyme with loss of activity.

Advantages of oral insulin in comparison with the injection commercial forms are evident, since long everyday injections could provoke various serious complications: they are accompanied with pain syndrome; lead to development of lipodystrophy, being not only cosmetic defects but also causing need in increase of hormone doses; traumatize psychics, especially one of children, provokes stress conditions, leading to the more expressed hyperglycemia, that, in turn, increases need in hormone etc.

The insulin in the form of an oral preparation, being water-in-oil microemulsion consisting of insulin, lipids and protease inhibitor is known. Then microemulsion is coated with carboxymetilcellulose (Y. W. Cho, M. Flynm, Lancet, 1989, 30, p. 1518)

The essential disadvantage of this preparation along with the labour-intensive and expensive technology of manufacturing is use of carboxymetilcellulose as carriers. It is subjected to microbe impact especially in conditions of industrial production: In addition, cellulose is capable to absorb the substantial quantities of protein which is necessary to wash out with high ionic force buffer. Carrying out of such procedure in large scales is expensive and could lead to considerable inactivation of insulin.

The method for producing insulin in the form of an oral preparation by means of incubation of insulin with erythrocytes, taken in ratio of 1-4:100 in presence of multifunctional binding agent, with final concentration being in range 0.15-0.25% is known. Usually erythrocytes isolated from cattle, pig or human blood are used as carriers one, and mainly bromic cyan, cyanur chloride or glutaraldialdegide are used as binding agent (RF Patent no. 2058788, Cl. A 61 K 38/28, issued Apr. 27, 1996). The preparation is emulsified in water before use.

A disadvantage of the known method is high toxicity of binding agents and necessity of expensive purification of final product required by the method.

The method for producing insulin in the form of an oral preparation by means of immobilization of insulin in volume of sewn polymer modified with inhibitor of proteolytic enzymes is known (R. Z. Creenley, et. all Polymer Matrices for oral delivery, Polymer Preprits 1990, V. 31, N 2, p. 182-183). Acrylic or metacrylic acids sewn by triethyleneglicol—di(met) acrylate are used as sewn polymer, and aprothenin—pancreatic inhibitor of trypsin is used as inhibitor of proteolytic enzymes.

A disadvantage of this method is low stability of obtained preparation to action of digestive enzyme, which result is low activity of insulin penetrating in blood.

The most closed to the claimed method—prototype is the method of obtaining of insulin preparation in form of gel by means of immobilization of insulin in volume of sewn polymer, modified with inhibitor of proteolytic enzymes, as which one uses ovomucoid from egg protein in concentration of 0.2-25 mg/g (hydrogel swelled in water). Immobilization is carried out by means of immersion of sewn modified polymer in aqueous solution of insulin with concentration of 0.01-5 mg/ml for 1-2 hours up to total swelling of polymer. Modified polymer is used in quantity of 0.01-1.0 g for 1 ml of insulin solution (RF Patent no. 2066551, Cl. A 61 K 38/28, issued Sep. 20, 1996).

A disadvantage of the known method is technologic complexity of isolation of ovomucoid and obtaining of sewn polymer, being modified by it, expensiveness and low therapeutic efficiency of obtained preparation.

### Essence of invention

The technical task of proposed invention is simplification and reduction of prices of the method for producing insulin in the form of an oral preparation as well as increasing of its therapeutic efficiency by means of immobilization of insulin on aqueous-soluble polymer. Determined difference of the claimed method as compared with the prototype is that insulin is modified by a polymer being activated by ionizing irradiation that allows to simplify the method and to increase therapeutic efficiency of preparation. At irradiation high active carbonyl groups are formed in polymer in process of radiation—chemical oxidation. The polymer activated by such way forms an aqueous-soluble complex with insulin, which decreases efficiently glucose level at oral intake. In consequence of high solubility in aqueous solutions, the complex of insulin with polymer is soaked in blood in full without diffusion limitations. Examples of polymers include but not limited to, dextranes, polyvinilpirrolidons, isoprenols, polyacrylamid, polyurethane.

### THE BEST VARIANCE FOR EMBODIMENT OF THE INVENTION

Technical task is reached by the proposed method consisting in following.

One uses 1-50% aqueous solution of polyethylenoxide with molar mass from 0.4 to 40 kDa. Then the solution is irradiated with high energy ionizing radiation, mainly by gamma-radiation or accelerated electron beam in doses providing carrying out of free-radical reactions, mainly 1.0-5.0 Mrad. Then insulin is added in solution of radiation-activated polyethylenoxide up to final concentration (by protein) from 1-10 mg/ml (or by insulin activity 10-100 ME/ml accordingly), in polyethylenoxide:insulin ratio equal to (1-500): 1, the mixture is stirred in 10-30 minutes up to obtaining of homogeneous transparent or slightly opalescent solution.

Use of ovomucoid as protease inhibitor in the prototype-method allows to protect insulin from proteolytic enzymes, and polyacrylamid gel executes function of depositing of modified insulin. At particular penetration in blood through intestine walls insulin is not deposed purposefully in liver because has not affinity to reticuloendothelial system organs and in connection with this its action will be similar to action of insulin when parenterally administered. To the contrary, insulin, modified by radiation-activated polymer, shows properties of basal insulin, i.e. its pharmacological effect is approached maximal to physiological mechanism. It is promoted by the polymer, having ability to be captured by liver cells.

Obtained technical result was not evident from known scientific-technical data of properties of named polymers and insulin since in result of insulin modification with radiation activated polymer it could loss completely its specific hypoglycemic activity owing to changing of its conformation and interaction with insulin cellular receptor. However the claimed method allows not only to keep specific hypoglycemic activity of insulin preparation but and provides maximal effective physiological mechanism of its action owing to that activated polymer carrier forms with insulin chemically labile connection and is used only as transporting carrier of insulin in liver cells where release of native insulin takes place.

### EXAMPLES

The invention is illustrated by following examples of concrete obtaining of insulin preparation.

### Example 1

10% aqueous solution of polyethylenoxide with molar mass of 1.5 kDa is irradiated with accelerated electron beam in dose of 5.0 Mrad. Insulin is added in the irradiated solution up to final concentration of 10 mg in 1 ml (polyethylenoxide:insulin ratio is 10:1). The mixture is stirred in 10 minutes and insulin preparation is obtained in form of slightly opalescent solution. Yield of finished product is 98%.

### Example 2

50.0% aqueous solution of polyethylenoxide with molar mass of 0.4 kDa is irradiated with braked gamma-radiation in dose of 1.0 Mrad. Insulin is added in the irradiated solution up to final concentration of 1 mg in 1 ml (polyethylenoxide:insulin ratio is 500:1). The mixture is stirred in 30 minutes and insulin preparation is obtained in form of transparent solution. Yield of finished product is 97%.

### Example 3

5% aqueous solution of polyethylenoxide with molar mass of 15 kDa is irradiated with accelerated electron beam in dose of 2.5 Mrad. Insulin is added in the irradiated solution up to final concentration 10 mg in 1 ml (polyethylenoxide:insulin ratio is 5:1). The mixture is stirred in 15 minutes and insulin preparation is obtained in form of slightly opalescent solution. Yield of finished product is 99%.

Results of testing of hypoglycemic action of insulin complex with radiated-activated polyethylenoxide on intact rats of Wistar line is presented in Table 1. In the test group 1 ml of pig insulin (composition: 30 ME/ml of pig insulin, 12.5 mass % of radiated—activated polyethylenoxide 1500, polyethylenoxide:insulin ratio is 125:1) modified with polyethylenoxide has been momentary inserted intragastric in test animals. In control group 1 ml of pig insulin with activity 30 ME/ml has been momentary inserted intragastric in animals.

TABLE 1

| | Time of measurement of glucose level in blood, min | | | | | | | | | | |
|---|---|---|---|---|---|---|---|---|---|---|---|
| | 0 | 60 | 120 | 180 | 240 | 300 | 360 | 420 | 480 | 540 | 600 |
| | Glucose concentration in blood, mmol/l | | | | | | | | | | |
| Animal No. | | | | | | | | | | | |
| (Test) | | | | | | | | | | | |
| 1 | 7.9 | 5.1 | 5.6 | 5.3 | 5.1 | 5.1 | 4.6 | 3.6 | 5.4 | 5.7 | 5.9 |
| 2 | 5.9 | 5.3 | 5.0 | 4.8 | 4.3 | 4.7 | 4.9 | 4.4 | 4.9 | 6.1 | 5.9 |
| 3 | 5.2 | 5.2 | 6.3 | 5.1 | 4.7 | 4.6 | 4.3 | 4.3 | 5.2 | 6.1 | 4.9 |
| 4 | 7.1 | 7.1 | 6.2 | 5.7 | 4.9 | 4.6 | 4.7 | 4.9 | 5.9 | 4.8 | 4.6 |
| 5 | 6.1 | 6.0 | 6.7 | 6.0 | 6.3 | 6.2 | 4.7 | 5.6 | 6.3 | 5.6 | 5.1 |
| Animal no. | | | | | | | | | | | |
| of | | | | | | | | | | | |
| (Cintrol) | | | | | | | | | | | |
| 1 | 6.5 | 5.3 | 7.3 | 5.6 | 5.6 | 5.4 | 5.2 | 5.0 | 6.6 | 6.1 | 5.6 |
| 2 | 6.8 | 6.2 | 7.4 | 6.4 | 6.1 | 5.9 | 5.9 | 5.3 | 5.6 | 5.4 | 5.1 |
| 3 | 5.3 | 5.8 | 5.5 | 5.7 | 5.1 | 5.3 | 6.2 | 5.4 | 4.8 | 5.5 | 5.4 |
| 4 | 7.5 | 6.7 | 7.7 | 6.8 | 5.5 | 5.4 | 6.3 | 7.6 | 6.4 | 6.0 | 5.2 |
| 5 | 5.9 | 6.9 | 6.1 | 6.2 | 6.7 | 6.1 | 5.5 | 5.2 | 6.1 | 5.8 | 5.8 |
| Average | 6.44 | 5.74 | 5.96 | 5.38 | 5.06 | 5.04 | 4.64 | 4.56 | 5.54 | 5.66 | 5.28 |
| value | | | | | | | | | | | |
| (Test) | | | | | | | | | | | |
| Average | 6.40 | 6.18 | 6.80 | 6.14 | 5.80 | 5.62 | 5.82 | 5.70 | 5.90 | 5.76 | 5.42 |
| value | | | | | | | | | | | |
| (Control) | | | | | | | | | | | |

As it is seen from results presented in Table 1, insulin modified with polyethylenoxide begins to show the main hypoglycemic activity in 3 hours after intragastric insertion and keeps it up to 10 hours. Obtained data testify approach of modified insulin action to basal secretion of insulin by pancreas.

Data of testing of hypoglycemic activity of human genetic engineering insulin modified with polyethylenoxide on rat model of alloxan diabetes are presented in Table 2. In the test group 1 ml of insulin (composition: 50 ME/ml of human insulin, 12.5 mass % of radiated—activated polyethylenoxide 1500, polyethylenoxide:insulin ratio is 70:1) modified with polyethylenoxide has been momentary inserted intragastric in the test animals. In control group 1 ml of human insulin with activity 50 ME/ml has been momentary inserted intragastric in animals.

TABLE 2

| | | Time of measurement of glucose level in blood, min | | | | | |
|---|---|---|---|---|---|---|---|
| Test | Initial | 2 | 4 | 6 | 8 | 10 | 24 |
| series | value | Change of glucose concentration in blood, mmol/l | | | | | |
| Control | 23.4 ± 2.8 | −1.1 ± 2.1 | −1.2 ± 1.7 | −5.1 ± 2.3 | −0.4 ± 4.8 | +1.3 ± 3.8 | −0.8 ± 1.8 |
| (n = 5) | | | | | | | |
| Test | 26.2 ± 3.5 | −5.8 ± 1.0 | −9.1 ± 1.0 | −8.9 ± 1.5 | −4.9 ± 3.2 | −0.5 ± 2.3 | −4.1 ± 2.2 |
| (n = 5) | | | | | | | |

As it is seen from presented results, modified insulin has expressed hypoglycemic activity at intragastric insertion in rat model of alloxan diabetes.

Comparative data of influence of insulin modified with polyethylenoxide (polyethylenoxide: insulin ration is 70:1) on absolute values of insulin level in blood plasma of rats at momentary intragastric insertion is presented in Table 3. In the test group human insulin modified with polyethylenoxide has been inserted intragastricly in intact rats of Wistar line with mass 200-240 g from calculation 250 ME/kg. Equivalent quantity of non-modified insulin has been inserted intragastrically in animals in control group. Content of insulin in blood plasma of rats (in μE/ml) has been defined by imunnoenzyme method.

TABLE 3

| | | Time of measurement of insulin content in blood | | |
|---|---|---|---|---|
| | | plasma of rats, hours | | |
| Test | Initial | 4 | 5 | 6 |
| series | values | Content of insulin in blood plasma of rats (in μE/ml) | | |
| Intact | 26 ± 7 | 16 ± 9 | 14 ± 7 | 16 ± 8 |
| animals | | | | |
| Control | 27 ± 10 | 10 ± 3 | 15 ± 6 | 17 ± 10 |
| Test | 23 ± 6 | 23 ± 6 | 35 ± 10 | 20 ± 6 |

It is seen from data presented in Table that insulin modified with polyethylenoxide at intragastric insertion either leads to increase of insulin level in blood plasma or prevents its decrease, i.e. modified insulin is approached maximally to basal insulin secreted by pancreas in the frames of physiological norm.

The claimed method for producing insulin in the form of an oral preparation as distinct from prototype-method is distinguished with simplicity and efficiency since its obtaining consists only of two stages in which one uses polymer carrier—polyethylenoxide, technologically simple method of its activation—action of ionizing radiation on polymer solution—as well as simple method of modification of insulin with activated polymer by means of addition of insulin in activated polymer solution up to required concentration (activity). Insulin preparation obtained by claimed method has high therapeutic activity being defined both by decrease of glucose concentration in blood and by direct determination of insulin level in blood.

1. The method for producing insulin in the form of an oral preparation including mixing of the last with aqueous soluble polymer being activated previously by means of irradiation with ionizing radiation.

2. The method according to claim 1, wherein polymer is polyethylenoxide with molar mass 0.4-40 kDa and concentration in range of 1.0-50.0%, and insulin—polyethylenoxide ratio is 1: (1-500) up to final insulin concentration in mixture of 1-10 mg/ml.

3. The method according to claim 1, wherein polyethylenoxide is activated by beam of accelerated electrons or gamma-radiation in doses of 1.0-5.0 Mrad.
